(19) United States
(12) Patent Application Publication
(10) Pub. No.: US 2013/0172536 A1
(43) Pub. Date: Jul. 4, 2013

(54) Intravenous Cytomegalovirus Human Immune Globulin and Manufacturing Method Thereof

(71) Applicants: Yunjia Zhang, Shenzhen (CN); Caiping Guo, Shenzhen (CN); Yujiang Ding, Shenzhen (CN); Qingshuang Song, Shenzhen (CN); Xin Zhang, Shenzhen (CN); Jincai Wang, Shenzhen (CN); Enying Wu, Shenzhen (CN); Pei Zhang, Shenzhen (CN); Yuqin Chen, Shenzhen (CN); Hui Li, Shenzhen (CN); Weirong Huang, Shenzhen (CN); Meilan Dai, Shenzhen (CN); Yanpeng Zhang, Shenzhen (CN); Chunhua Wang, Shenzhen (CN); Shan Luo, Shenzhen (CN); Qiaoyun Huang, Shenzhen (CN); Yongdi Liu, Shenzhen (CN); Kaiyong Wu, Shenzhen (CN)

(72) Inventors: Yunjia Zhang, Shenzhen (CN); Caiping Guo, Shenzhen (CN); Yujiang Ding, Shenzhen (CN); Qingshuang Song, Shenzhen (CN); Xin Zhang, Shenzhen (CN); Jincai Wang, Shenzhen (CN); Enying Wu, Shenzhen (CN); Pei Zhang, Shenzhen (CN); Yuqin Chen, Shenzhen (CN); Hui Li, Shenzhen (CN); Weirong Huang, Shenzhen (CN); Meilan Dai, Shenzhen (CN); Yanpeng Zhang, Shenzhen (CN); Chunhua Wang, Shenzhen (CN); Shan Luo, Shenzhen (CN); Qiaoyun Huang, Shenzhen (CN); Yongdi Liu, Shenzhen (CN); Kaiyong Wu, Shenzhen (CN)

(73) Assignee: SHENZHEN WEIGUANG BIOLOGICAL PRODUCTS CO.,LTD., Shenzhen,Guangdong (CN)

(21) Appl. No.: 13/821,991

(22) PCT Filed: Aug. 16, 2011
(86) PCT No.: PCT/CN11/78485

§ 371 Date: Mar. 10, 2013

(51) Int. Cl. C07K 1/36; B65B 1/04; C07K 16/08

(52) U.S. Cl. CPC C07K 1/36; C07K 16/088; B65B 1/04
USPC 530/389.4; 53/425

(57) ABSTRACT

The present invention discloses an intravenous cytomegalovirus human immune globulin and a manufacturing method thereof, wherein the technical problem to be solved is to improve the purity, yield, and safety of the product. The intravenous cytomegalovirus human immune globulin of the present invention has a specific activity of no less than 2.5 PEI-U/mg, an anti-CMV titer of no less than 100 PEI-U/ml, a purity of greater than 98.2%, and a protein content of 51˜55 mg/ml. The present invention employs caprylic acid precipitation and anion exchange chromatography for replacing the step of ethanol precipitation in the conventional cold ethanol method to keep IgG in the supernatant and maintain the activity of the IgG; the present invention employing processes of caprylic acid inactivation of virus and nanometer film virus removal can effectively protect the safety of the product, and studies show that the preparing method of the present invention not only improves the purity, yield, and safety of the product; but also saves energy and reduces the cost of production.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention relates to a human immune globulin and a manufacturing method thereof, and more particularly to an intravenous cytomegalovirus human immune globulin and a manufacturing method thereof.

2. Description of Related Arts

Cytomegalovirus are a DNA virus of Betaherpesvirinae, which results in a mortality rate of up to 50%˜80% in pregnant women, newborn babies, organ transplant patients, and immunosuppressed patients who are infected with this virus. An intravenous cytomegalovirus human immune globulin, CMV-IgG, can specifically neutralize the cytomegalovirus, which is mainly used for treating pregnant women, newborn babies, immunosuppressed patients, and organ transplant patients who are infected with cytomegalovirus. The natural infection rate of cytomegalovirus in the general population is over 80%, and the intravenous cytomegalovirus human immune globulin prepared by specific isolation and purification methods from plasma containing high titer of anti-CMV IgG antibodies has irreplaceable clinical value for the treatment of the patients with severe infection caused by cytomegalovirus, wherein the plasma is collected from healthy people who were infected with cytomegalovirus.

Currently, most kinds of intravenous cytomegalovirus human immune globulins contained in the listed drugs are isolated and purified by the cold ethanol method (Edwin J. Cohn, L. E. Strong, W. L. Hughes JR., D. J. Mulford, J. N. Ashworthm, M. Melin and H. L. Taylor, J. Am. Chem. Soc., 68 (1946) 459-475.), which was invented by professor Edwin J. Cohn from Harvard University in U. S in 1946 and can be used for isolating different proteins by adjusting the five parameters of the pH, the protein concentration, the temperature, the concentration of ethanol, and the ionic strength in the process. It has been proved by the long-term practice that the cold ethanol method has the following disadvantages: ethanol is a protein denaturant and is able to cause structural changes of IgG resulting in denaturation and inactivation of IgG during the separation, lead to a low recovery rate of potency, and may also bring about new epitopes. Additionally, compared to column chromatographic techniques, the ethanol precipitation purification method has a low efficiency and usually needs to reduce the protein recovery to meet the requirement of purification of products, so the process results in a low recovery of product and low purification of product. Lastly, the isolation process generally must be carried out at low temperatures, which causes defects in high cost hardware, high running cost, and high labor intensity.

### SUMMARY OF THE PRESENT INVENTION

An object of the present invention is to provide an intravenous cytomegalovirus human immune globulin and a manufacturing method thereof, wherein the technical problems to be solved are to improve the purity, yield, and safety of the product.

The present invention employs the following technical solution: An intravenous cytomegalovirus human immune globulin, wherein said intravenous cytomegalovirus human immune globulin has a specific activity of no less than 2.5 PEI-U/mg, an anti-CMV titer of no less than 100 PEI-U/ml, a purity of greater than 98.2%, and a protein content of 51˜55 mg/ml.

A method for preparing intravenous cytomegalovirus human immune globulin, comprising the steps of:

(1) preparing FI+II+III, FII+III deposits

preparing human plasma measured by enzyme linked immunosorbent assay, and

dissolving said human plasma at 2˜30° C., and mixing said human plasma, wherein the plasma has a high titer of anti-CMV,

preparing FI+II+III deposit

adjusting the protein content of said human plasma to 45˜55 mg/ml with saline, adjusting the pH of said human plasma to 6.0˜6.5 with glacial acetic acid, and adding 95% ethanol to adjust the ethanol concentration of said human plasma to 20˜25%, wherein the reaction temperature is −5.5˜−4.5° C., then the human plasma is stirred for 4˜6 hours, and after the reaction is completed, obtaining FI+II+III deposit by centrifuging or pressure filtering,

(2) preparing FI+II+III deposit

adjusting the protein content of said human plasma to 45˜55 mg/ml with saline, adjusting the pH of said human plasma to 6.8˜7.2 with glacial acetic acid, adding 95% ethanol to adjust the ethanol concentration of said human plasma to 7.5˜8.5%, wherein the reaction temperature is −2.5˜−2.0° C., then the human plasma is stirred for 4 hours, and after the reaction is completed the FI deposit are removed by centrifuging or pressure filtering and the supernatant is obtained. The FII+III deposits are obtained by adjusting the pH of said supernatant to 6.0˜6.5 with glacial acetic acid, adding 95% ethanol to adjust the ethanol concentration to 20˜25%, wherein the reaction temperature is −5.5˜−4.5° C., then the supernatant is stirred for 4˜6 hours, and after the reaction is completed the FII+III deposit are obtained by centrifuging or pressure filtering;

(3) dissolving FI+II+III or FII+III deposits

adding 20˜80 mM sodium acetate buffer solution and stirring for 8˜16 hours at 2˜8° C. to dissolve completely the FI+II+III or FII+III deposit, and isolating the supernatant by centrifuging or pressure filtering, wherein the sodium acetate buffer solution has a pH of 4.8˜5.2 and the volume of the sodium acetate buffer solution is 0.9˜1.1 times the plasma;

(4) depositing with caprylic acid

adjusting the pH of the supernatant to 4.5˜5.5 with 4 mol/L acetic acid or 0.5˜1 mol/L sodium hydroxide, adding caprylic acid until the concentration of caprylic acid is 10˜100 mmol/L, and isolating the supernatant by centrifuging or filtering;

(5) inactivating virus with caprylic acid

filtering the supernatant with a 1.0 μm filter membrane, controlling the pressure to no more than 0.25 MPa, adjusting the pH of the filtrate to 4.5˜5.5 with 4 mol/L acetic acid or 0.5˜1 mol/L sodium hydroxide, adding water for injection or caprylic acid to adjust the concentration of caprylic acid of the suspension to 20˜80 mmol/L, stirring for 1˜2 hours at 20˜30° C., and then isolating the supernatant by centrifuging or filtering;

(6) depositing with ethanol

adjusting the pH of the supernatant to 4.5˜5.5 with 0.5˜1 mol/L hydrochloric acid or sodium hydroxide, adding 95% ethanol until the concentration of ethanol is 12˜16%, stirring for 2˜8 hours at −4.0˜−2.5° C., and isolating the supernatant by centrifuging or pressure filtering;

(7) ultrafiltering

filtering the supernatant by 0.45 μm filter membrane, controlling the pressure to no more than 0.25 MPa, concentrating the filtrate with 30 KD ultrafiltration membranes by 15˜20 times, ultrafiltering and dialyzing with 20˜60 mmol/L phosphate buffer solution. After ultrafiltering, controlling, and obtaining a product of solution containing the protein content of 25˜40 mg/ml, wherein the phosphate buffer solution has a pH of 6.0˜7.1 and the volume of the phosphate buffer solution is 8˜10 times the supernatant;

(8) purifying with anion exchange chromatography

using a 20˜60 mmol/L phosphate buffer solution as the equilibration buffer solution to balance the chromatography column, wherein the phosphate buffer solution has a pH of 6.0˜7.1 and the volume of the phosphate buffer solution is 8˜10 times the volume of the chromatography column, and calculating the amount of protein of the sample loaded with no more than 70˜80% of the maximum loading capacity per ml filler. After loading and collecting the penetrated solution and eluting with a 20˜60 mmol/L phosphate buffer solution to remove other protein hung in the chromatography column, wherein the phosphate buffer solution contains 2 mol/L NaCl and has a pH of 6.0˜7.1;

(9) filtering with nano film filter to remove virus

adjusting the pH of the penetrated solution to 4.2˜5.0 by using 0.5˜1 mol/L HCl as the eluting solution after pre-filtering with a 0.1 μm filter membrane, and then removing virus by filtering with Novasip DV20 nano film filter and controlling the pressure to no more than 0.25 Mpa;

(10) ultrafiltering

after removing virus, the filtrate is concentrated by filtering with 30 KD ultrafiltration membranes until the protein content of the filtrate is 80˜100 mg/ml, then ultrafiltering with water for infection, wherein the water for infection is 8˜10 times the filtrate, and after ultrafiltering and controlling a product of solution containing the protein content of 80˜150 mg/ml is obtained;

(11) preparing

measuring the protein content of the ultrafiltrated filtrate and adjusting the protein content of the product to 51˜55 mg/ml with water for infection, adding maltose malt sugar until the content of maltose malt sugar is 9˜11%, and adjusting the pH to 3.8˜4.2 with 0.5˜1 mol/L hydrochloric acid. The method of the present invention comprises the steps for sterilizing and packing after preparing, wherein the sterilization is via a 0.2 μm membrane, the pressure during the filtering is controlled to no more than 0.25 Mpa, and the packaging specifications are that the protein content is 51˜55 mg/ml and the titer is no less than 100 PEI-U/ml.

The method of the present invention further comprises the steps of sampling and measuring the quality indicators of the protein content, the anti-CMV titer, the purity, the range of distribution of molecular weight, the amount of residue of caprylic acid, and the osmolality of the packed product.

The filling material is added in the process of the anion exchange chromatography according to the present invention, wherein the filling material is selected from the group consisting of DEAE Sepharose Fast Flow, TOYOPEARL DEAE 650M, and Macro-Prep DEAE Media.

Compared with the process of cold ethanol fractionation of the prior art, the present invention has the following technical effects:

(1) employing the processes of caprylic acid precipitation and anion exchange chromatography, which have mild reaction conditions and get rid of the cold ethanol fractionation step, wherein the method of the present invention improves the rate of production, and at the same time effectively maintains the activity of IgG and improves the purity and rate of recovery. The rate of recovery and potency of the process is 40˜65%, the purification fold is 5.30˜8.14, the rate of recovery of IgG is no less than 4.9 g/L, the purity is no less than 98.2%, and the IgG multimer is not more than 0.1%.

(2) employing the processes of caprylate virus inactivation and nano-membrane filtration for removal of viruses, which can effectively inactivate and remove viruses, wherein when combining with anion exchange chromatography, the drop in amount of virus is greater than 12 log 10.

(3) caprylic acid can effectively precipitate proteins of no-immunoglobulin impurities, IgG, IgA, and ceruloplasmin that remained in the supernatant; when precipitated, the recovery rate of titer of antibody is at least 90%.

(4) the process of anion exchange chromatography can effectively remove polymers and acidic protein impurities such that the final product doesn't contain impurities such as polymer and albumin

(5) the process of the present invention has a production cycle of 5˜7 days, in comparison to the prior art of cold ethanol process which has a production cycle of 28˜30 days. The process of the present invention effectively improves the production efficiency, and reduces the ethanol usage, energy consumption, labor intensity, and cost of production.

The method for preparing intravenous cytomegalovirus human immune globulin of the present invention not only improves the purity, yield, and safety of the product, but also reduces energy consumption and cost of production.

### BRIEF DESCRIPTION OF THE DRAWINGS

FIG. 1 is a flow diagram of the process for purifying the intravenous cytomegalovirus human immune globulin of the present invention.

FIG. 2 is a flow diagram of the process of the cold ethanol method for preparing general human immune globulins according to the prior art.

FIG. 3 is a view of the electrophoresis pattern of the intravenous cytomegalovirus human immune globulin by polyacrylamide gel electrophoresis according to a first preferred embodiment of the present invention.

FIG. 4 is a view of the electrophoresis pattern of the intravenous cytomegalovirus human immune globulin by polyacrylamide gel electrophoresis according to a second preferred embodiment of the present invention.

FIG. 5 is a view of the electrophoresis pattern of the intravenous cytomegalovirus human immune globulin by polyacrylamide gel electrophoresis according to a third preferred embodiment of the present invention.

FIG. 6 is a view of the electrophoresis pattern of the intravenous cytomegalovirus human immune globulin by polyacrylamide gel electrophoresis according to a fourth preferred embodiment of the present invention.

FIG. 7 is a view of the electrophoresis pattern of the intravenous cytomegalovirus human immune globulin by polyacrylamide gel electrophoresis according to a fifth preferred embodiment of the present invention.

FIG. 8 is a view of the electrophoresis pattern of the intravenous cytomegalovirus human immune globulin by polyacrylamide gel electrophoresis according to a sixth preferred embodiment of the present invention.

### DETAILED DESCRIPTION OF THE PREFERRED EMBODIMENT

Referring to the drawings and the embodiments, the present invention is further described in detail as follows.

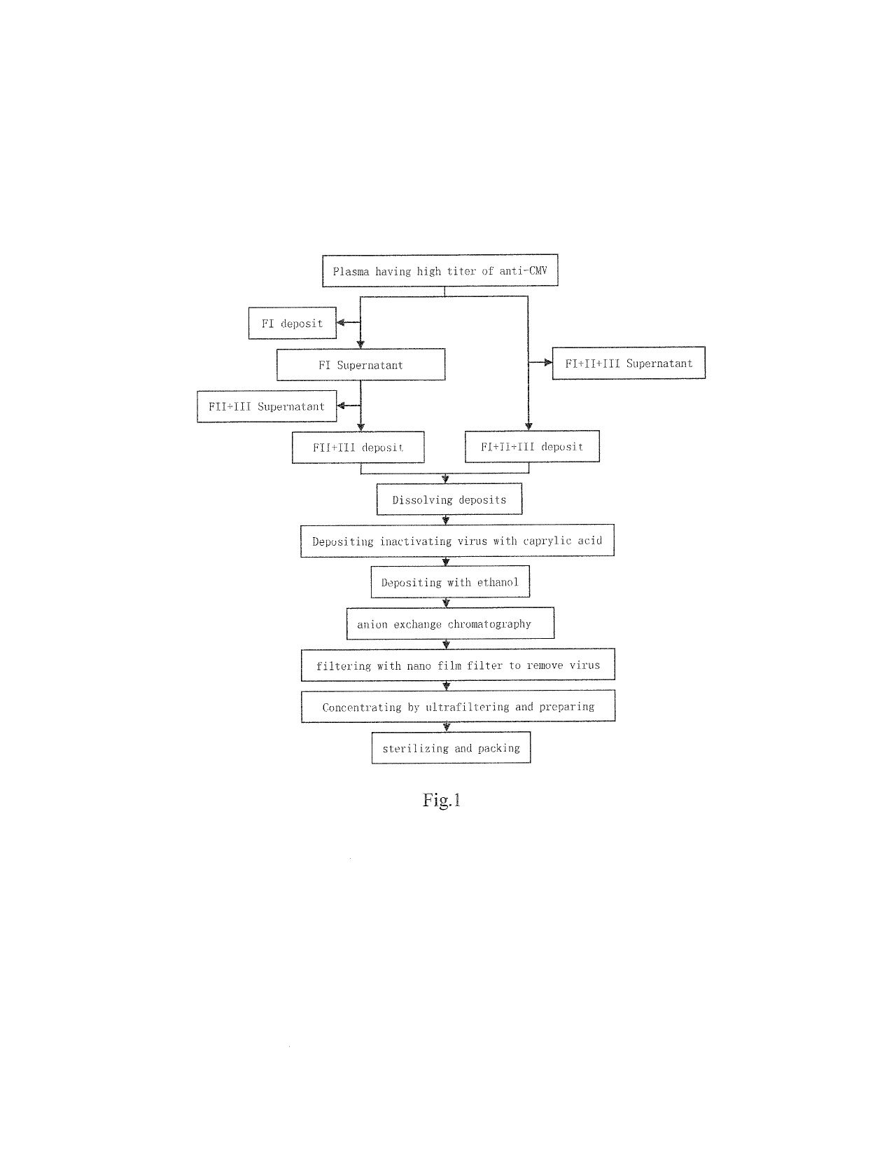

As shown in FIG. 1, the first embodiment of the present invention is described as the following steps:

(1) Preparing 20 units of human plasma measured by the enzyme-linked immunosorbent assay, and dissolving the human plasma at 25° C., wherein the human plasma has high titer of anti-CMV, and the volume of the mixture is 11540 ml.

(2) Adjusting the protein content of the human plasma to 49.53 mg/ml with 2310 ml saline, adjusting the pH of the human plasma to 6.18 with glacial acetic acid, and adding 3926 ml ethanol to adjust the ethanol concentration of the suspension of the human plasma to 22%, wherein the reaction temperature is adjusted to −5.0° C., then stirred for 4 hours, and after the reaction is completed, the FI+II+III deposit is obtained by centrifuging.

(3) Dissolving the FI+II+III deposit with 11500 ml sodium acetate buffer solution and stirring for 4 hours at 4° C. and then isolating the supernatant by centrifuging, wherein the sodium acetate buffer solution has a pH of 4.93 and a concentration of 50 mmol/L.

(4) Adjusting the pH of the supernatant to 4.57 with 4 mol/L acetic acid, adding caprylic acid until the concentration of the caprylic acid is 60 mmol/L, stirring for 3 hours at 21° C., and then isolating the supernatant by centrifuging.

(5) Filtering the supernatant with a 1.0 μm filter membrane, wherein the pressure during the filtering is controlled to no more than 0.25 Mpa and the pH to 4.74, wherein water of infection is added to adjust the concentration of the caprylic acid to 22 mmol/L, and then the solution is stirred for 1 hour at 22° C. to inactivate the enveloped virus, and then the supernatant is isolated by centrifuging.

(6) Adjusting the pH of the supernatant to 5.11 with 0.5 mol/L acetic acid, adding 2070 ml ethanol until the concentration of the ethanol is 14%/L, then left to react for 7 hours at −3.8° C., and then the supernatant is isolated by centrifuging.

(7) Filtering the supernatant with a 0.45 μm filter membrane, wherein the pressure during the filtering is controlled to no more than 0.25 Mpa, then concentrating the filtrate by 20 times with 30 KD ultrafiltration membranes, then ultra-filtering with the phosphate buffer solution which is 8 times of the filtrate, and then controlling and obtaining the 2000 ml product of solution containing the protein content of 36.81 mg/ml, wherein the phosphate buffer solution has a pH of 6.63 and a concentration of 25 mmol/L.

(8) Equalizing DEAE Sepharose Fast Flow (GE Healthcare Bio-Sciences AB, USA) column with 10 times the volume of the phosphate buffer solution which has a pH of 6.63 and a concentration of 25 mmol/L, wherein the volume of column is 200 ml, 7000 ml of penetrated solution is collected through the column by ultra-filtering the filtered filtrate, and the impurity hung in the column is eluted with a phosphate buffer solution containing 2 mol/L NaCl, wherein the phosphate buffer solution has a pH of 6.63 and a concentration of 25 mmol/L.

(9) Adjusting the pH of the penetrated solution to 4.25 with 1 mol/L HCl, after pre-filtering with a 0.1 μm filter membrane, wherein the virus is removed by filtering with Novasip DV20 nano film filter, wherein the pressure during the filtering is controlled to no more than 0.25 Mpa.

(10) After eliminating the virus, the filtered filtrate is concentrated by 10 times with 30 KD ultrafiltration membranes, and then ultrafiltering with water for infection; wherein the water for infection is 8 times of the filtrate, and then 600 ml raw filtrate is obtained, wherein the raw filtrate has a protein content of 107.21 mg/ml, then the filtrate is diluted with water of infection and maltose malt sugar is added until the content of maltose malt sugar is 10%, then the pH is adjusted to 4.02 with 0.5˜1 mol/L hydrochloric acid, and then the contents are sterilized by filtering with a 0.2 μm membrane, wherein the pressure during the filtering is controlled to no more than 0.25 Mpa, and then packed by the packaging specifications which are a protein content of 51˜55 mg/ml and a titer of no less than 100 PEI-U/ml. After packing, the protein content is sampled and measured by the Kjeldahl method, the titer by enzyme-linked immunosorbent assay, the purity of protein and the amount of residue of albumin by non reduced E SDS-PAGE method, the pH by methods for measuring pH, the amount of residue of caprylic acid by gas chromatography, and the osmolality by methods for measuring osmolality. Furthermore, the monomer, dimer, polymer, and cracking body of IgG are measured by HPLC, wherein the test results are shown in Table 6.

The recovery rate of titer and the recovery rate of protein in the process are as shown in Table 1.

TABLE 1
The recovery rates of titer and protein in the process

| | Content of | Titer of | Recovery of | Recovery | Specific activity | |
|---|---|---|---|---|---|---|
| | protein | anti-CMV | protein (g/L | of titer | (PEI-U/mg | Purification |
| Process | (mg/ml) | (PEI-U/ml) | plasma) | (%) | protein) | fold |
| Plasma | 59.45 | 20.79 | 59.45 | 100.00 | 0.35 | 1.00 |
| Dissolving of | 11.21 | 18.35 | 12.18 | 95.50 | 1.64 | 4.68 |
| FI + II + III | | | | | | |
| deposit | | | | | | |
| Inactivating | 7.39 | 16.12 | 8.53 | 83.86 | 2.18 | 6.23 |
| supernatant with | | | | | | |
| caprylic acid | | | | | | |
| Depositing | 4.90 | 12.24 | 6.78 | 75.13 | 2.50 | 7.14 |
| supernatant with | | | | | | |
| ethanol | | | | | | |
| Before | 32.73 | 82.58 | 6.38 | 68.54 | 2.52 | 7.21 |
| chromatography | | | | | | |
| Penetrating | 8.63 | 24.32 | 5.78 | 64.84 | 2.82 | 8.05 |
| After preparing | 51.08 | 128.17 | 5.56 | 62.97 | 2.85 | 8.14 |

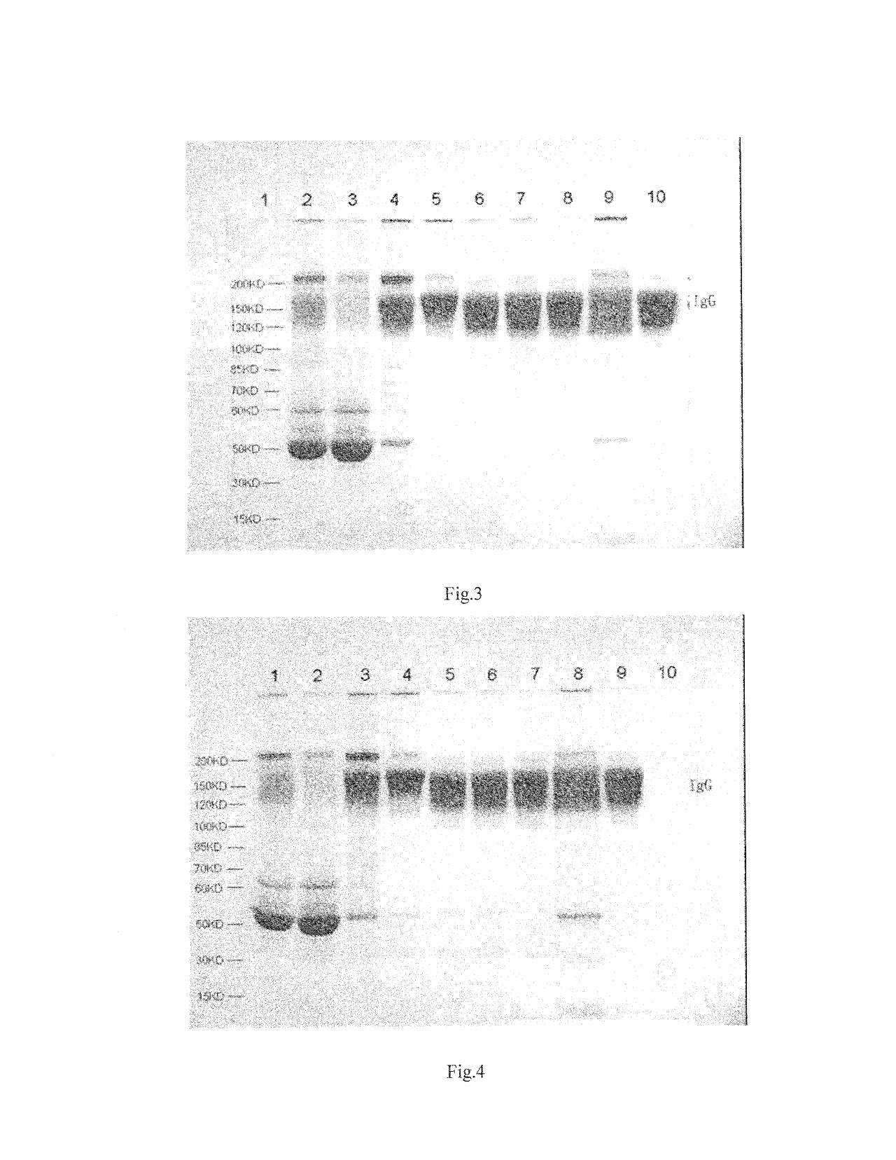

As shown in FIG. 3, the electrophoresis pattern of the intravenous cytomegalovirus human immune globulin according to the preferred embodiment of the present invention by polyacrylamide gel electrophoresis is shown, wherein the molecular weight of IgG is 150 KD˜160 KD, wherein reference symbols is as follows: 1, the loading buffer; 2, the plasma having high titer of anti-CM; 3, the FI+II+III supernatant; 4, the dissolved FI+II+III deposit; 5, the supernatant inactivated by caprylic acid; 6, the supernatant precipitated by ethanol; 7, the supernatant before DEAE chromatography; 8, the filtrated solution; 9, the eluted solution; 10, the prepared intravenous cytomegalovirus human immune globulin, CMV-IgG. The results of pattern analysis show the supernatant inactivated by caprylic acid has an 89.72% purity of IgG, and indicate that the caprylic acid precipitation and inactivation process can remove most of the impurity protein, such as albumin and fibrinogen. The electrophoretic band of the eluted solution shows anion exchange chromatography can effectively remove the impurities, such as multimer and residue of the albumin. The IgG in the filtrated solution has a purity of 98.76%.

The Second preferred embodiment comprises the following steps:

(1) Preparing 20 units of human plasma measured by the enzyme-linked immunosorbent assay, and dissolving the human plasma at 30° C., wherein the human plasma has high titer of anti-CMV, and the volume of the mixture is 11410 ml.

(2) Adjusting the protein content of the human plasma to 50.30 mg/ml with 2280 ml saline, adjusting the pH of the human plasma to 6.48 with glacial acetic acid, and adding 4603 ml ethanol to adjust the ethanol concentration of the suspension of the human plasma to 25%, wherein the reaction temperature is adjusted to −4.5° C., then stirred for 6 hours, and after the reaction is completed, the FI+II+III deposit is obtained by centrifuging;

(3) Dissolving the FI+II+III deposit with 10500 ml sodium acetate buffer solution and stirring for 12 hours at 2.2° C., and then isolating the supernatant by centrifuging; wherein the sodium acetate buffer solution has a pH of 5.16 and a concentration of 50 mmol/L.

(4) Adjusting the pH of the supernatant to 5.07 with 4 mol/L acetic acid, adding caprylic acid until the concentration of the caprylic acid is 30 mmol/L, stirring for 3 hours at 25° C., and then isolating the supernatant by centrifuging.

(5) Filtering the supernatant with a 1.0 μm filter membrane, wherein the pressure during the filtering is controlled to no more than 0.25 Mpa, adjusting the pH to 5.17 with 1 mol/L sodium hydroxide solution, and then adding caprylic acid to adjust the concentration of the caprylic acid to 55 mmol/L, then the supernatant is stirred for 1.5 hour at 25° C. to inactivate the enveloped virus, and then the supernatant is isolated by centrifuging.

(6) Adjusting the pH of the supernatant to 5.48 with 1 mol/L sodium hydroxide solution, adding 2190 ml ethanol until the concentration of the ethanol is 16%/L, the supernatant is then allowed to react for 4 hours at −3.0° C., and then the supernatant is isolated by centrifuging.

(7) Filtering the supernatant with a 0.45 μm filter membrane, wherein the pressure during the filtering is controlled to no more than 0.25 Mpa, then concentrating the filtrate by 15 times with 30 KD ultrafiltration membranes, and then ultra-filtering the filtrate with 8 times the phosphate buffer solution; after ultra-filtering, controlling and obtaining the 2000 ml product of solution containing a protein content of 33.55 mg/ml, wherein the phosphate buffer solution has a pH of 6.93 and a concentration of 50 mmol/L.

(8) Equalizing DEAE Sepharose Fast Flow column with 10 times the volume of the phosphate buffer solution having a pH of 6.93 and a concentration of 50 mmol/L, wherein the volume of column is 200 ml and 7000 ml penetrated solution is collected by filtering the ultra-filtered filtrate through a column, eluting the impurity hung in the column with a phosphate buffer solution containing 2 mol/L NaCl, wherein the phosphate buffer solution has a pH of 6.93 and a concentration of 50 mmol/L.

(9) Adjusting the pH of the penetrated solution to 5.07 with 1 mol/L HCl, after pre-filtering with a 0.1 μm filter membrane, and removing the virus by filtering with Novasip DV20 nano film filter, wherein the pressure during the filtering is controlled to no more than 0.25 Mpa.

After eliminating the virus, the filtrate is concentrated by 10 times by filtering with 30 KD ultrafiltration membranes, and then ultrafiltering with water for infection; wherein the water for infection is 8 times of the filtrate, and then 750 ml raw filtrate is obtained, wherein the raw filtrate has a protein content of 82.16 mg/ml mg/ml, then the filtrate is diluted with water of infection and maltose malt sugar is added until the concentration of maltose malt sugar is 10 g/L, then the pH is adjusted to 4.12 with 1 mol/L hydrochloric acid, and then the filtrate is sterilized by filtering with a 0.2 μm membrane, wherein the pressure during the filtering is controlled to no more than 0.25 Mpa, and then packed. After packing, sampling, and measuring the protein content, the titer, the purity of protein and the amount of residue of albumin, the pH, the monomer, the dimer, the polymer and the cracking body of IgG, the amount of residue of caprylic acid, and the osmolality by methods in the first embodiment of the present invention, wherein the test results are shown in Table 6.

The recovery rate of titer and the recovery rate of protein in the process are as shown in Table 2.

TABLE 2
The recovery rates of titer and protein in the process

| | Content of | Titer of | Recovery of | Recovery | Specific activity | |
|---|---|---|---|---|---|---|
| | protein | anti-CMV | protein (g/L | of titer | (PEI-U/mg | Purification |
| Process | (mg/ml) | (PEI-U/ml) | plasma) | (%) | protein) | fold |
| Plasma | 60.36 | 30.75 | 60.36 | 100.00 | 0.51 | 1.00 |
| Dissolving | 12.75 | 29.93 | 12.63 | 96.39 | 2.35 | 4.60 |
| FI + II + III | | | | | | |
| deposit | | | | | | |
| Inactivating | 8.73 | 26.86 | 8.42 | 84.21 | 3.08 | 6.03 |
| supernatant with | | | | | | |
| caprylic acid | | | | | | |
| Depositing | 4.97 | 19.16 | 6.27 | 70.99 | 3.86 | 7.56 |
| supernatant with | | | | | | |
| ethanol | | | | | | |
| Before | 29.49 | 114.75 | 5.88 | 63.12 | 3.89 | 7.63 |
| chromatography | | | | | | |
| Penetrating | 8.73 | 34.21 | 5.52 | 58.50 | 3.92 | 7.68 |
| After preparing | 51.78 | 191.37 | 5.37 | 55.09 | 3.70 | 7.25 |

As shown in FIG. 4, the electrophoresis pattern of the intravenous cytomegalovirus human immune globulin according to the second preferred embodiment of the present invention by polyacrylamide gel electrophoresis is shown, wherein the molecular weight of IgG is 150 KD-160 KD, wherein reference symbols is as follows: 1, the plasma having high titer of anti-CM; 2, the FI+II+III supernatant; 3, the dissolved FI+II+III deposit; 4, the supernatant inactivated by caprylic acid; 5, the supernatant precipitated by ethanol; 6, the supernatant before DEAE chromatography; 7, the filtrated solution; 8, the eluted solution; 9, the prepared intravenous cytomegalovirus human immune globulin, CMV-IgG; 10, the loading buffer. The electrophoresis pattern shows that the process of purification of the present embodiment has a the same purification effect as the first preferred embodiment, and the prepared IgG has a purity of 99.10%.

The third preferred embodiment comprises the steps of:

(1) Preparing 20 units of human plasma measured by the enzyme-linked immunosorbent assay, and dissolving the human plasma at 15° C., wherein the human plasma has high titer of anti-CMV and the volume of the mixture is 11570 ml.

(2) Adjusting the protein content of the human plasma to 49.69 mg/ml with 2310 ml saline, adjusting the pH of the human plasma to 6.32 with glacial acetic acid, and adding 4603 ml ethanol to adjust the ethanol concentration of the suspension of the human plasma to 20%, wherein the reaction temperature is adjusted to −4.8° C., then stirred for 6 hours, and after the reaction is completed, the FI+II+III deposit is obtained by centrifuging;

(3) Dissolving the deposit of FI+II+III with 12500 ml sodium acetate buffer solution and stirring for 14 hours at 6.0° C., and then isolating the supernatant by centrifuging; wherein the sodium acetate buffer solution has a pH of 5.02 and a concentration of 80 mmol/L.

(4) Adjusting the pH of the supernatant to 5.50 with 1 mol/L sodium hydroxide solution, adding caprylic acid until the concentration of the caprylic acid is 40 mmol/L, stirring for 1 hours at 23° C., and then isolating the supernatant by centrifuging.

(5) Filtering the supernatant with a 1.0 μm filter membrane, wherein the pressure during the filtering is controlled to no more than 0.25 Mpa, then adjusting the pH to 5.50 with 1 mol/L sodium hydroxide solution, then adding caprylic acid to adjust the concentration of the caprylic acid to 78 mmol/L, then stirring for 1 hour at 30° C. to inactivate enveloped virus, and then isolating the supernatant by centrifuging.

(6) Adjusting the pH of the supernatant to 4.62 with 1 mol/L hydrochloric acid, adding 95% ethanol until the concentration of ethanol is 12%, stirring for 3 hours at −2.5° C., and then isolating the supernatant by centrifuging.

(7) Filtering the supernatant with a 0.45 μm filter membrane, wherein the pressure during the filtering is controlled to no more than 0.25 Mpa, then concentrating by 18 times the filtrate with 30 KD ultrafiltration membranes, then ultra-filtering with 10 times the phosphate buffer solution of as the filtrate, and after ultra-filtering, controlling and obtaining the 2000 ml product of solution containing the protein content of 34.54 mg/ml, wherein the phosphate buffer solution has a pH of 6.87 and a concentration of 40 mmol/L.

(8) Equalizing DEAE Sepharose Fast Flow column with the phosphate buffer solution having a pH of 6.87 and a concentration of 40 mmol/L, wherein the volume of column is 200 ml and 6000 ml penetrate solution is collected by filtering the ultra-filtered filtrate through a column, eluting the impurity hung in the column with a phosphate buffer solution containing 2 mol/L NaCl, wherein the phosphate buffer solution has a pH of 6.87 and a concentration of 40 mmol/L.

(9) Adjusting the pH of the penetrated solution to 4.62 with 1 mol/L HCl, after pre-filtering with a 0.1 μm filter membrane, and removing the virus by filtering with Novasip DV20 nano film filter, wherein the pressure during the filtering is controlled to no more than 0.25 Mpa.

(10) After eliminating the virus, the filtered filtrate is concentrated 10 times with 30 KD ultrafiltration membranes, and then ultrafiltering with water for infection; wherein the water for infection is 8 times of the filtrate, and then 500 ml raw filtrate is obtained, wherein the raw filtrate has a protein content of 126.57 mg/ml, then the filtrate is diluted with water of infection and maltose malt sugar is added until the content of maltose malt sugar is 10 g/L, then the pH is adjusted to 3.85 with 1 mol/L hydrochloric acid, and then the contents are sterilized by filtering with a 0.2 μm membrane, wherein the pressure during the filtering is controlled to no more than 0.25 Mpa. After packing, sampling, and measuring the protein content, the titer, the purity of protein, the pH, the monomer, the dimer, the polymer, and the cracking body of IgG, the amount of residue of albuminthe, amount of residue of caprylic acid, and the osmolality by methods in the first embodiment of the present invention, wherein the test results are shown in Table 6.

The recovery rate of titer and the recovery rate of protein in the process are as shown in Table 3.

TABLE 3
The recovery rates of titer and protein in the process

| | Content of | Titer of | Recovery of | Recovery | Specific activity | |
|---|---|---|---|---|---|---|
| | protein | anti-CMV | protein (g/L | of titer | (PEI-U/mg | Purification |
| Process | (mg/ml) | (PEI-U/ml) | plasma) | (%) | protein) | fold |
| Plasma | 59.61 | 27.83 | 59.61 | 100.00 | 0.47 | 1.00 |
| FI + II + III | | | | | | |
| Dissolving | 11.82 | 24.40 | 12.27 | 90.99 | 2.06 | 4.39 |
| FI + II + III | | | | | | |
| deposit | | | | | | |
| Inactivating | 8.65 | 20.71 | 8.88 | 76.41 | 2.39 | 5.09 |
| supernatant with | | | | | | |
| caprylic acid | | | | | | |
| Depositing | 4.93 | 13.68 | 6.31 | 61.26 | 2.77 | 5.90 |
| supernatant with | | | | | | |
| ethanol | | | | | | |
| Before | 35.97 | 100.56 | 5.92 | 58.40 | 2.80 | 5.95 |
| chromatography | | | | | | |
| Penetrating solu | 9.77 | 28.62 | 5.67 | 53.33 | 2.93 | 6.23 |
| After preparing | 52.23 | 155.47 | 5.46 | 50.70 | 2.98 | 6.33 |

As shown in FIG. 5, the electrophoresis pattern of the intravenous cytomegalovirus human immune globulin according to the third preferred embodiment of the present invention by polyacrylamide gel electrophoresis is shown, wherein the molecular weight of IgG is 150 KD˜160 KD, wherein reference symbols are as follows: 1, the loading buffer; 2, the plasma having high titer of anti-CM; 3, the FI+II+III supernatant; 4, the dissolved FI+II+III deposit; 5, the supernatant inactivated by caprylic acid; 6, the supernatant precipitated by ethanol; 7, the supernatant before DEAE chromatography; 8, the filtrated solution; 9, the prepared intravenous cytomegalovirus human immune globulin, CMV-IgG; 10, the eluted solution; 11, the prepared intravenous cytomegalovirus human immune globulin, CMV-IgG; 12, the prepared intravenous cytomegalovirus human immune globulin, CMV-IgG; 13, the prepared intravenous cytomegalovirus human immune globulin, CMV-IgG; 14, the listed human immune globulin, CMV-IgG (Abroad); and 15, the loading buffer. The electrophoresis pattern shows the referring example of the listed intravenous human immune globulin at abroad has a purity of 86.6%, which contains 2.2% polymer, 7.52% dimer and 2.51% albumin, and the process of purification of the present embodiment has the same purification effect as the first preferred embodiment, and the prepared IgG has a purity of 98.89%.

The fourth preferred embodiment comprises the steps of:

(1) Preparing 20 units of human plasma measured by the enzyme-linked immunosorbent assay, and dissolving the human plasma at 4° C., wherein the human plasma has high titer of anti-CMV and the volume of the mixture is 11670 ml.

(2) Adjusting the protein content of the human plasma to 49.27 mg/ml with 2330 ml saline, then adjusting the pH of the human plasma to 6.02 with glacial acetic acid, and adding 4494 ml 95% ethanol to adjust the ethanol concentration of the suspension of the human plasma to 23%, wherein the reaction temperature is adjusted to −5.5° C., and then stirred for 6 hours, and after the reaction is completed, the FI+II+III deposit is obtained by centrifuging.

(3) Dissolving the FI+II+III deposit with 12000 ml sodium acetate buffer solution and stirring for 10 hours at 8.0° C., then isolating the supernatant by centrifuging; wherein the sodium acetate buffer solution has a pH of 4.81 and a concentration of 30 mmol/L.

(4) Adjusting the pH of the supernatant to 4.96 with 1 mol/L sodium hydroxide solution, adding caprylic acid until the concentration of the caprylic acid is 10 mmol/L, stirring for 3 hours at 18° C., and then isolating the supernatant by centrifuging.

(5) Filtering the supernatant with a 1.0 μm filter membrane, wherein the pressure during the filtering is controlled to no more than 0.25 Mpa, then adjusting the pH to 5.21 with 1 mol/L sodium hydroxide solution, then adding caprylic acid to adjust the concentration of the caprylic acid to 46 mmol/L, then the contents are stirred for 2 hour at 27° C. to inactivate enveloped virus, and then the supernatant is isolated by centrifuging.

(6) Adjusting the pH of the supernatant to 5.04 with 1 mol/L hydrochloric acid, adding 2396 ml of 95% ethanol until the concentration of ethanol is 15%, then the contents are stirred for 5 hours at −3.5° C., and then the supernatant is isolated by centrifuging.

(7) Filtering the supernatant with a 0.45 μm filter membrane, wherein the pressure during the filtering is controlled to no more than 0.25 Mpa, then concentrating the filtrate by 20 times with 30 KD ultrafiltration membranes, then ultra-filtering with the phosphate buffer solution which is 10 times that of the filtrate, and after ultra-filtering, controlling and obtaining 2000 ml of product solution containing the protein content of 37.17 mg/ml, wherein the phosphate buffer solution has a pH of 7.02 and a concentration of 60 mmol/L.

(8) Equalizing Macro-Prep DEAE Media (Bio-Rad, USA) column with 10 times the volume of the phosphate buffer solution having a pH of 7.02 and a concentration of 60 mmol/L, wherein the volume of column is 200 ml, and 8000 ml of penetrated solution is collected by filtering the ultra-filtered filtrate through the column, eluting the impurity hung in the column with a phosphate buffer solution containing 2 mol/L NaCl, wherein the phosphate buffer solution has a pH of 7.02 and a concentration of 60 mmol/L.

(9) Adjusting the pH of the penetrated solution to 4.47 with 1 mol/L HCl, after pre-filtering with a 0.1 μm filter membrane, and removing the virus by filtering with Novasip DV20 nano film filter, wherein the pressure during the filtering is controlled to no more than 0.25 Mpa.

(10) After eliminating the virus, the filtrate is concentrated 10 times by filtering with 30 KD ultrafiltration membranes, and then ultrafiltering with water for infection, wherein the water for infection is 8 times that of the filtrate, then 500 ml raw filtrate is obtained, wherein the raw filtrate has a protein content of 134.27 mg/ml, and then the contents are diluted with water of infection and maltose malt sugar is added until the content of maltose malt sugar is 11 g/L, then the pH is adjusted to 4.08 with 1 mol/L hydrochloric acid, and then sterilized by filtering with a 0.2 μm membrane, wherein the pressure during the filtering is controlled to no more than 0.25 Mpa. After packing, sampling, and measuring the protein content, the titer, the purity of protein and the amount of residue of albumin, the pH, the monomer, the dimer, the polymer and the cracking body of IgG, the amount of residue of caprylic acid, the osmolality by methods in the first embodiment of the present invention, wherein the test results are shown in Table 6.

The recovery rate of titer and the recovery rate of protein in the process are as shown in Table 4.

TABLE 4
The recovery rates of titer and protein in the process

| | Content of | Titer of | Recovery of | Recovery | Specific activity | |
|---|---|---|---|---|---|---|
| | protein | anti-CMV | protein (g/L | of titer | (PEI-U/mg | Purification |
| Process | (mg/ml) | (PEI-U/ml) | plasma) | (%) | protein) | fold |
| Plasma | 59.11 | 21.60 | 59.11 | 100.00 | 0.37 | 1.00 |
| Dissolving | 12.17 | 18.60 | 13.07 | 92.50 | 1.53 | 4.13 |
| FI + II + III | | | | | | |
| deposit | | | | | | |
| Inactivating | 8.36 | 16.54 | 9.41 | 80.39 | 1.98 | 5.34 |
| supernatant with | | | | | | |
| caprylic acid | | | | | | |
| Depositing | 4.87 | 11.47 | 6.86 | 68.24 | 2.36 | 6.37 |
| supernatant with | | | | | | |
| ethanol | | | | | | |
| Before | 36.29 | 87.70 | 6.37 | 66.98 | 2.42 | 6.53 |
| chromatography | | | | | | |
| Penetrating | 8.09 | 20.51 | 6.02 | 65.10 | 2.54 | 6.85 |
| After preparing | 52.81 | 140.20 | 5.74 | 61.19 | 2.65 | 7.18 |

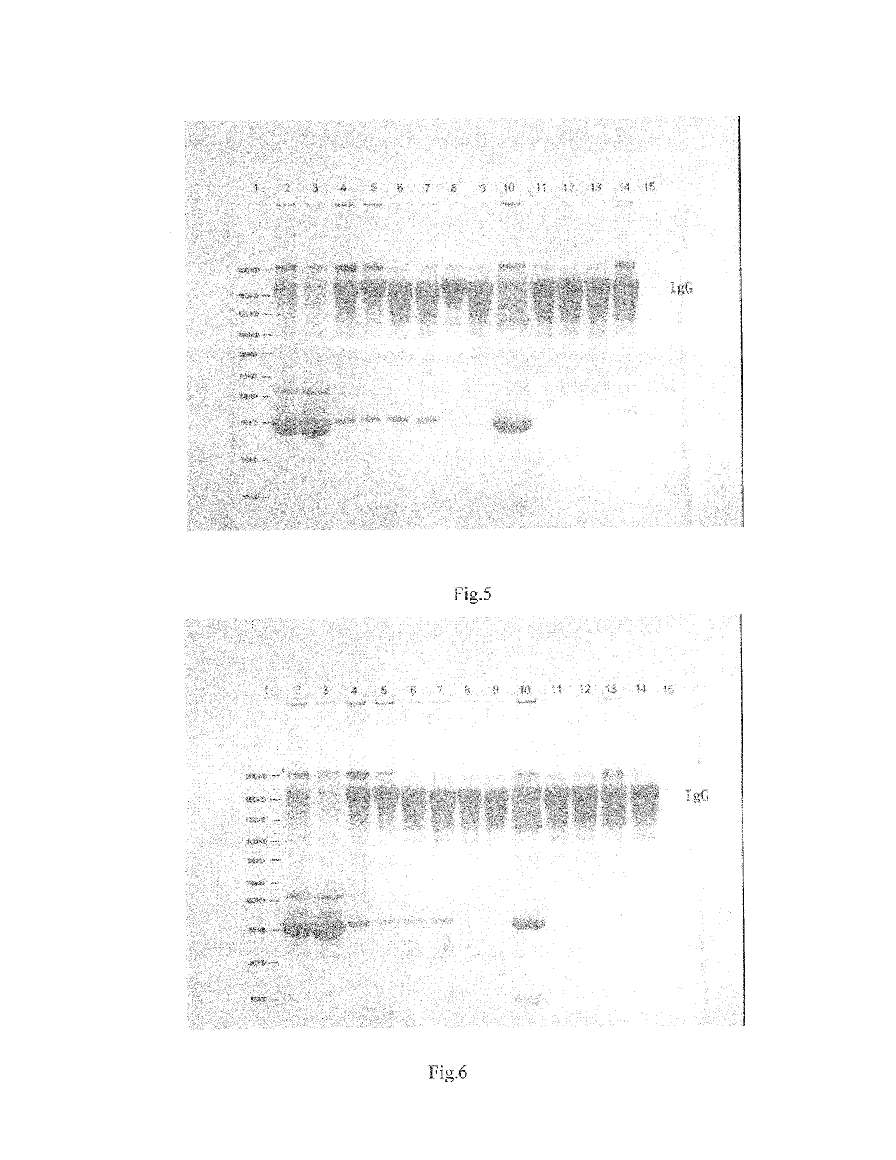

As shown in FIG. 6, the electrophoresis pattern of the intravenous cytomegalovirus human immune globulin according to the fourth preferred embodiment of the present invention by polyacrylamide gel electrophoresis is shown, wherein the molecular weight of IgG is 150 KD˜160 KD, wherein reference symbols are as follows: 1, the loading buffer; 2, the plasma having high titer of anti-CM; 3, the FI+II+III supernatant; 4, the dissolved FI+II+III deposit; 5, the supernatant inactivated by caprylic acid; 6, the supernatant precipitated by ethanol; 7, the supernatant before DEAE chromatography; 8, the filtrated solution; 9, the prepared intravenous cytomegalovirus human immune globulin, CMV-IgG; 10, the eluted solution; 11, the prepared intravenous cytomegalovirus human immune globulin, CMV-IgG; 12, the prepared intravenous cytomegalovirus human immune globulin, CMV-IgG; 13, the listed human immune globulin (Abroad); 14, the listed human immune globulin (Abroad); and 15, the loading buffer. Comparing the purity of the domestic intravenous cytomegalovirus human immune globulin with the purity of the foreign listed intravenous cytomegalovirus human immune globulin by the electrophoresis pattern, the results show the domestic intravenous cytomegalovirus human immune globulin contains 95.38% IgG, 2.37% dimer, 0.8% polymer, and 0.52% albumin. The process of purification of the present embodiment has the same purification effect as the first preferred embodiment and the prepared IgG has a purity of 98.27%.

The differences between a fifth embodiment and the above 1-4 embodiments are that the plasma is first precipitated by 8% ethanol to separate the FI deposit, and then is precipitated by 20% ethanol to prepare FII+III deposit for subsequent purification, in addition, the filling of TOYOPEARL DEAE 650M (TOSOH, Japanese) is used for replacing the filling of DEAE Sepharose Fast Flow.

The Fifth Embodiment Comprises the Steps of:

(1) Preparing 2 units of human plasma measured by the enzyme-linked immunosorbent assay, and dissolving the human plasma at 20° C., wherein the human plasma has high titer of anti-CMV and the volume of the mixture is 1150 ml.

(2) Adjusting the protein content of the plasma to 50.72 mg/ml with 230 ml saline, the pH to 7.00 with glacial acetic acid, the concentration of ethanol of the suspension with 130 ml of 95% ethanol to 8%, the reaction temperature to −2.5° C., and then the contents are stirred for 4 hours, after the reaction is finished, the FI supernatant is separated by centrifuging, the pH is adjusted to 6.25, 247 ml of 95% ethanol is added to adjust the concentration of ethanol of the suspension to 20%, then the contents are allowed react for 6 hours at 5.0° C., and then the FII+III deposit is separated by centrifuging when the reaction is finished.

(3) Dissolving the FII+III deposit with 1150 ml sodium acetate buffer solution, then stirring for 12 hours at 3.0° C., and then separating the supernatant by centrifuging; wherein the sodium acetate buffer solution has a pH of 4.98 and a concentration of 40 mmol/L.

(4) Adjusting the pH of the supernatant to 4.78 with 4 mol/L acetic acid, adding caprylic acid until the concentration of the caprylic acid is 100 mmol/L, the contents are then stirred for 2 hours at 22° C., and then the supernatant is isolated by centrifuging.

(5) Filtering the supernatant with a 1.0 μm filter membrane, controlling the pressure to no more than 0.25 Mpa, adjusting the pH to 5.08 with 0.5 mol/L sodium hydroxide, adding water of infection to adjust the concentration of caprylic acid to 38 mmol/L, then the contents are stirred for 1 hour at 23° C. to inactivate enveloped virus, and then the supernatant is isolated by centrifuging.

(6) Adjusting the pH of the suspension to 5.03, adding 208 ml of 95% ethanol until the concentration of ethanol is 14%, then the contents are stirred for 8 hours at −4.0° C., and then the supernatant is isolated by centrifuging.

(7) Filtering the supernatant with a 0.45 μm filter membrane, controlling the pressure to no more than 0.25 Mpa, concentrating the filtrate by 15 times with 30 KD ultrafiltration membranes, ultra-filtering with the phosphate buffer solution 10 times that of the filtrate, and after ultra-filtering, obtaining the 170 ml of product solution containing the protein content of 38.09 mg/ml, wherein the phosphate buffer solution has a pH of 6.46 and a concentration of 25 mmol/L.

(8) Equalizing DEAE Sepharose Fast Flow column with the phosphate buffer solution having a pH of 6.46 and a concentration of 25 mmol/L, wherein the volume of the phosphate buffer solution is 10 times that of the volume of column, and the volume of column is 22 ml, wherein 328 ml of penetrated solution is collected by filtering the ultra-filtered filtrate through the column, eluting the impurity hung in the column with the phosphate buffer solution containing 2 mol/L NaCl, wherein the phosphate buffer solution has a pH of 6.46 and a concentration of 25 mmol/L.

(9) Adjusting the pH of the penetrated solution to 4.75 with 1 mol/L HCl, after pre-filtering with a 0.1 μm filter membrane, and removing the virus by filtering with Novasip DV20 nano film filter, wherein the pressure during the filtering is controlled to no more than 0.25 Mpa.

(10) After eliminating the virus, the filtrate is concentrated 10 times by filtering with 30 KD ultrafiltration membranes, and then ultrafiltering with water for infection, wherein the water for infection is 8 times that of the filtrate, then 50 ml raw filtrate is obtained, wherein the raw filtrate has a protein content of 115.23 mg/ml, then the filtrated is diluted with water of infection and maltose malt sugar is added until the content of maltose malt sugar is 9 g/L, and then adjusting the pH to 3.98 with 1 mol/L hydrochloric acid, and then the contents are sterilized by filtering with a 0.2 μm membrane, wherein the pressure during the filtering is controlled to no more than 0.25 Mpa. After packing, sampling, and measuring the protein content, the titer, the purity of protein and the amount of residue of albumin, the pH, the monomer, the dimer, the polymer and the cracking body of IgG, the amount of residue of caprylic acid, and the osmolality by methods in the first embodiment of the present invention, wherein the test results are shown in Table 6.

The recovery rate of titer and the recovery rate of protein in the process are as shown in Table 5.

TABLE 5
The recovery rates of titer protein in the process

| | Content of | Titer of | Recovery of | Recovery | Specific activity | |
|---|---|---|---|---|---|---|
| | protein | anti-CMV | protein (g/L | of titer | (PEI-U/mg | Purification |
| Process | (mg/ml) | (PEI-U/ml) | plasma) | (%) | protein) | fold |
| Plasma | 60.86 | 32.42 | 60.86 | 100.00 | 0.53 | 1.00 |
| Dissolving | 13.45 | 25.37 | 14.15 | 82.34 | 1.89 | 3.56 |
| FI + II + III | | | | | | |
| deposit | | | | | | |
| Inactivating | 9.98 | 19.63 | 10.32 | 62.66 | 1.97 | 3.71 |
| supernatant with | | | | | | |
| caprylic acid | | | | | | |
| Depositing | 5.62 | 13.41 | 6.79 | 50.00 | 2.39 | 4.50 |
| supernatant with | | | | | | |
| ethanol | | | | | | |
| Before | 36.00 | 95.21 | 5.63 | 45.97 | 2.64 | 4.99 |
| chromatography | | | | | | |
| Penetrating | 15.79 | 43.07 | 5.24 | 44.13 | 2.73 | 5.15 |
| After preparing | 51.52 | 144.75 | 4.92 | 42.71 | 2.81 | 5.30 |

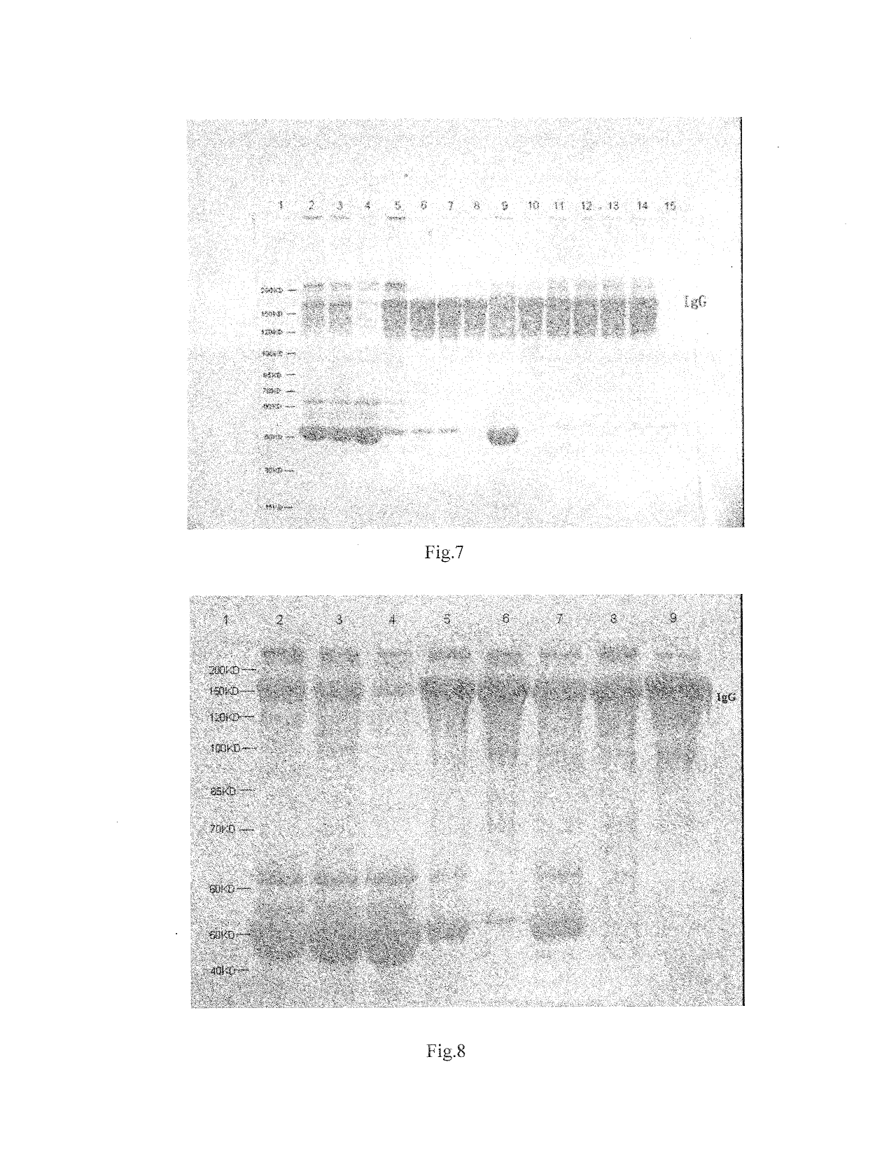

As shown in FIG. 7, the electrophoresis pattern of the intravenous cytomegalovirus human immune globulin according to the fifth preferred embodiment of the present invention by polyacrylamide gel electrophoresis is shown, wherein reference symbols are as follows: 1, the loading buffer; 2, the plasma having high titer of anti-CM; 3, the FI supernatant; 4, the FII+III supernatant 5, the dissolved FII+III deposit; 6, the supernatant precipitated by ethanol; 7, the supernatant before DEAE chromatography; 8, the prepared intravenous cytomegalovirus human immune globulin, CMV-IgG; 9, the eluted solution; 10, the listed human immune globulin (Domestic); 11, the listed human immune globulin (Abroad); 12, the listed human immune globulin (Abroad); 13, the listed human immune globulin (Abroad); 14, the listed human immune globulin (Abroad); and 15, the loading buffer. The electrophoresis pattern shows the process of purification of the present embodiment has the same purification effect as the first embodiment, and the prepared IgG has a purity of 99.82%.

### Example 1

### For Comparing

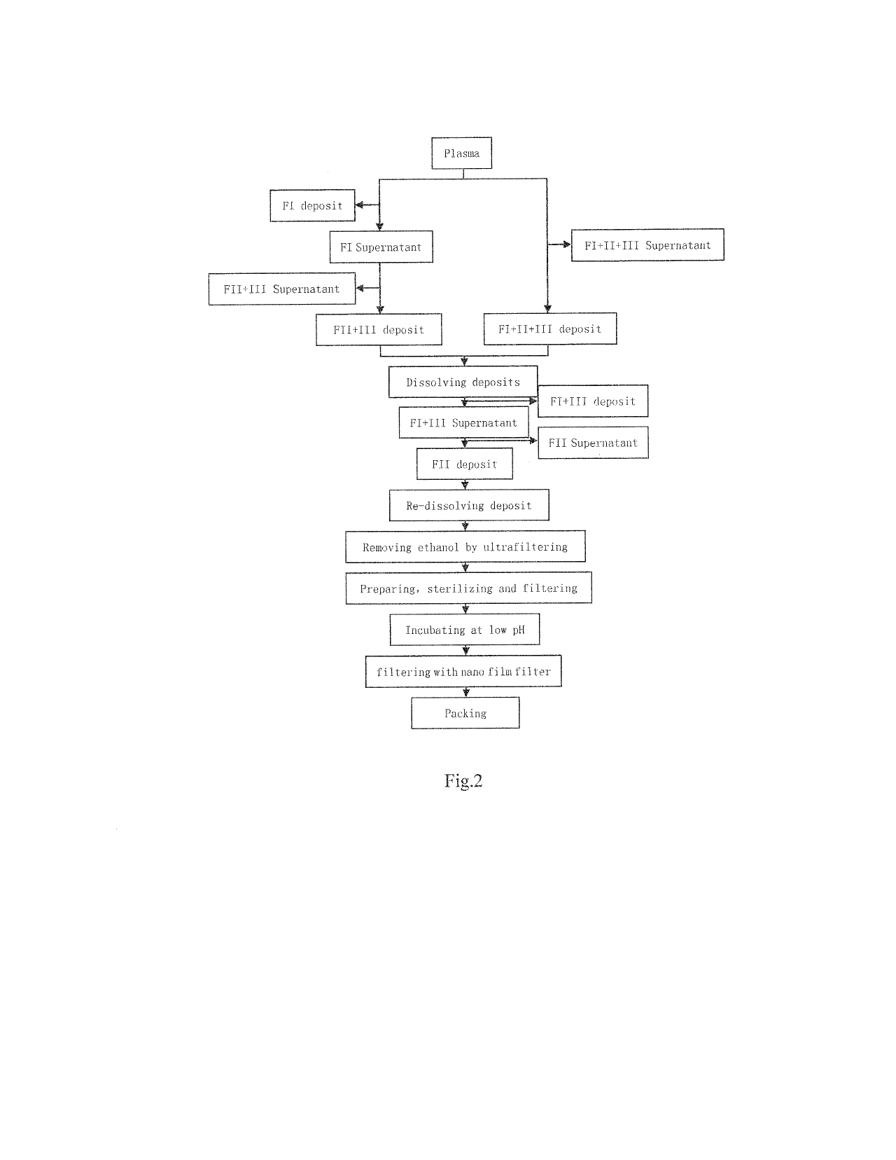

The present example employs the cold ethanol method of the prior art for preparing general immune globulin (named in Pharmacopoeia: intravenous human immune globulin pH4), as shown in FIG. 2, comprising the steps as follows:

(1) preparing 5 units of plasma from general population, dissolving the plasma at 20° C., and mixing the dissolved plasma to obtain 2850 ml mixture.

(2) adjusting the protein content of the plasma to 47.36 mg/ml with 640 ml saline, adjusting the pH to 6.28 with glacial acetic acid, adding 930 ml ethanol to adjust the concentration of ethanol of the suspension to 20%, adjusting the reaction temperature to −4.5° C., then the contents are stirred for 4 hours, and after the reaction is finished, the FI+II+III deposit is separated by centrifuging.

(3) dissolving the FI+II+III deposit with 2300 ml of 20 mmol/L sodium acetate buffer solution, then stirring for 4 hours at 4° C., and then separating the supernatant by centrifuging; wherein the sodium acetate buffer solution has a pH of 5.07.

(4) adjusting the pH of the suspension to 5.25, adding 456 ml of 95% ethanol to adjust the concentration of ethanol to 15%, then allowing the contents to react for 3 hours at −3.5° C., and then the FI+III supernatant is separated by centrifuging.

(5) adjusting the pH of the suspension to 7.05 with 1 mol/L sodium hydroxide solution, adding 205 ml of 95% ethanol to adjust the concentration of ethanol to 20%, the contents are then allowed to react for 6 hours at −7.0° C., and then the FII deposit is separated by centrifuging.

(6) dissolving the FII deposit with water of infection, then stirring for 12 hours at 2˜8° C., and then separating the supernatant by centrifuging.

(7) concentrating the supernatant to 100 ml with 30 KD ultrafiltration membrane, removing ethanol with water of infection, obtaining 150 ml example of solution, wherein the volume of the water of infection is 10 times that of the supernatant.

(8) preparing no less than 50 mg/ml protein and 10 g/L maltose, adjusting the pH to 3.97 with 1 mol/L hydrochloric acid, sterilizing with a 0.2 μm filter membrane, and controlling the pressure for filtering to no more than 0.25 Mpa,

(9) after sterilizing, retaining the prepared example in a viral inactivation room, incubating for 21 days at 23˜25° C., after incubating, removing the virus by filtering with Novasip DV20 nano film filter, controlling the pressure for filtering to no more than 0.25 Mpa. After packing, sampling, and measuring the content of protein, the titer, the purity of protein, the pH, the monomer, the dimer, the polymer and the cracking body of IgG, the amount of residue of albumin, and the osmolality by methods in the first embodiment of the present invention. The test results are shown in Table 6.

The quality indicators of examples of the prepared intravenous cytomegalovirus human immune globulin

| Project | Embodiment 1 | Embodiment 2 | Embodiment 3 | Embodiment 4 | Embodiment 5 | Example 1 |
|---|---|---|---|---|---|---|
| Content of protein | 51.08 | 51.78 | 52.23 | 52.81 | 51.52 | 51.58 |
| (mg/ml) | | | | | | |
| Titer (PEI-U/ml) | 128.17 | 191.37 | 155.47 | 140.20 | 144.75 | — |
| Purity (%) | 98.76 | 99.10 | 98.89 | 98.27 | 99.82 | 96.7% |
| pH | 4.02 | 4.12 | 3.85 | 4.08 | 3.98 | 3.97 |
| Monomer of IgG (%) | 99.33 | 99.45 | 99.28 | 99.18 | 99.46 | 95.33% |
| Dimer (%) | 0.62 | 0.52 | 0.67 | 0.74 | 0.47 | 2.52% |
| Polymer (%) | 0.05 | 0.03 | 0.05 | 0.08 | 0.07 | 1.17% |
| Cracking body (%) | 0.00 | 0.00 | 0.00 | 0.00 | 0.00 | 0.46 |
| Albumin (%) | 0.00 | 0.00 | 0.00 | 0.00 | 0.00 | 0.52 |
| Residue of caprylic acid | <50 | <50 | <50 | <50 | <50 | — |
| (μmol/L) | | | | | | |
| Osmolality | 326 | 337 | 316 | 342 | 307 | 331 |
| (mOsmol/kg) | | | | | | |

As shown in FIG. 8, the electrophoresis pattern of the intravenous cytomegalovirus human immune globulin according to the example 1 for comparing of the present invention by polyacrylamide gel electrophoresis is shown, wherein 1, the loading buffer; 2, the plasma from general population; 3, the dissolved plasma; 4, the FI+II+III supernatant; 5, the dissolved FI+II+III deposit; 6, the FI+III supernatant; 7, the dissolved FI+II+III deposit; 8, the dissolved FII deposit; and 9, the prepared intravenous cytomegalovirus human immune globulin, CMV-IgG. The electrophoresis pattern shows that the purity of IgG of the prepared intravenous cytomegalovirus human immune globulin is 96.7%.

What is claimed is:

1. An intravenous cytomegalovirus human immune globulin having a specific activity of no less than 2.5 PEI-U/mg, an anti-CMV titer of no less than 100 PEI-U/ml, a purity of greater than 98.2%, and a protein content of 51˜55 mg/ml.

2. A method for preparing intravenous cytomegalovirus human immune globulin, comprising the steps of:
(a) preparing FI+II+III and FII+III deposits:
preparing human plasma measured by enzyme linked immunosorbent assay, dissolving said human plasma at 2˜30° C., and mixing said human plasma, wherein said human plasma has a high titer of anti-CMV;
preparing FI+II+III deposit;
adjusting the protein content of said human plasma to 45˜55 mg/ml with saline, adjusting the pH of said human plasma to 6.0˜6.5 with glacial acetic acid, and adding 95% ethanol or ethanol to adjust the concentration of ethanol of said human plasma to 20˜25%, wherein the reaction temperature is −5.5˜−4.5° C., and then stirred for 4˜6 hours, after the reaction is finished, obtaining FI+II+III deposit by centrifuging or pressure filtering;
preparing FI+II+III deposit;
adjusting the protein content of said human plasma to 45˜55 mg/ml with saline, adjusting the pH of said human plasma to 6.8˜7.2 with glacial acetic acid, and adding 95% ethanol or ethanol to adjust the concentration of ethanol of said human plasma to 7.5˜8.5%, wherein the reaction temperature is −2.5˜−2.0° C., and then stirred for 4 hours, after the reaction is finished, removing FI deposit by centrifuging or pressure filtering to obtain the supernatant, adjusting the pH of said supernatant to 6.0˜6.5 with glacial acetic acid, adding 95% ethanol or ethanol to adjust the concentration of ethanol of said supernatant to 20˜25%, wherein the reaction temperature is −5.5˜−4.5° C., and then stirred for 4˜6 hours, after the reaction is finished, obtaining FII+III deposit by centrifuging or pressure filtering;
(b) dissolving FI+II+III or FII+III deposit:
dissolving FI+II+III or FII+III deposit with 20˜80 mM sodium acetate buffer solution, stirring for 8˜16 hours at 2˜8° C. to dissolve completely FI+II+III or FII+III deposit, separating the supernatant by centrifuging or pressure filtering, wherein the pH of said sodium acetate buffer solution is 4.8˜5.2, and the amount of said sodium acetate buffer solution is 0.9˜1.1 times said plasma;
(c) depositing with caprylic acid:
adjusting the pH of said supernatant to 4.5˜5.5 with 4 mol/L acetic acid or 0.5˜1 mol/L sodium hydroxide, adding caprylic acid until the concentration of caprylic acid is 10˜100 mmol/L, stirring for 1˜3 hours at 18˜25° C., and separating the supernatant by centrifuging or filtering;
(d) inactivating virus with caprylic acid:
filtering said supernatant with a 1.0 μm filter membrane, controlling the pressure to no more than 0.25 MPa, adjusting the pH of the filtrate to 4.5˜5.5 with 4 mol/L acetic acid or 0.5˜1 mol/L sodium hydroxide, adding water for infection or caprylic acid to adjust the concentration of caprylic acid of the suspension to 20˜80 mmol/L, stirring for 1˜2 hours at 20˜30° C., and separating the supernatant by centrifuging or filtering;
(e) depositing with ethanol:
adjusting the pH of said supernatant to 4.5˜5.5 with 0.5˜1 mol/L hydrochloric acid or sodium hydroxide, adding 95% ethanol or ethanol for precipitating until the concentration of ethanol is 12˜16%, stirring for 2˜8 hours at −4.0˜−2.5° C., and separating the supernatant by centrifuging or pressure filtering;
(f) ultrafiltering:
filtering said supernatant with a 0.45 μm filter membrane, controlling the pressure to no more than 0.25 MPa, concentrating the filtrate with 30 KD ultrafiltration membrane by 15˜20 times, ultrafiltering and dialyzing with 20˜60 mmol/L phosphate buffer solution, after ultrafiltering, controlling and obtaining a solution product containing 25˜40 mg/ml protein, wherein said phosphate buffer solution has a pH of 6.0˜7.1, and the volume of said phosphate buffer solution is 8˜10 times of said supernatant;
(g) purifying with anion exchange chromatography:
using a phosphate buffer solution as an equilibration buffer solution to balance the chromatography column, wherein said phosphate buffer solution has a pH of 6.0˜7.1, a concentration of 20˜60 mmol/L, wherein the volume of said phosphate buffer solution is 8˜10 times the volume of said chromatography column, calculating the amount of protein of the sample loaded to no more than 70˜80% of the maximum loading capacity per ml filler, after loading, collecting the penetrated solution and eluting with a 20˜60 mmol/L phosphate buffer solution having a pH of 6.0˜7.1 to remove other protein hung in said chromatography column, wherein said phosphate buffer solution contains 2 mol/L NaCl;
(h) filtering with nano film filter to remove virus:
adjusting the pH of the penetrated solution to 4.2˜5.0 with 0.5˜1 mol/L HCl, after pre-filtering with a 0.1 μm filter membrane, and removing the virus by Novasip DV20 nano film filter, controlling the pressure to no more than 0.25 Mpa;
(i) Ultrafiltering:
after removing the virus, adjusting the protein content of the filtrate to 80˜100 mg/ml by ultrafiltering with 30 KD ultrafiltration membranes, ultrafiltering with water for infection, wherein the water for infection is 8˜10 times of said filtrate, and after ultrafiltering, controlling and obtaining a product of solution containing the protein content of 80˜150 mg/ml; and
(j) preparing:
measuring the protein content of the ultrafiltrated filtrate, adjusting the protein content of product to 51˜55 mg/ml with water of infection, adding maltose malt sugar until the content of maltose malt sugar is 9˜11%, and adjusting the pH to 3.8˜4.2 with 0.5˜1 mol/L hydrochloric acid.

3. The method for preparing intravenous cytomegalovirus human immune globulin, as recited in claim 2, comprising the steps of sterilizing and packing after preparing, wherein the step of sterilizing is carried out by a 0.2 μm filter membrane and the pressure during the filtering is controlled to no more than 0.25 Mpa, and the packaging specifications comprise the protein content of 51˜55 mg/ml and the titer of no less than 100 PEI-U/ml.

4. The method for preparing intravenous cytomegalovirus human immune globulin, as recited in claim 3, further comprising the steps of sampling and measuring the quality indicators of the protein content, the anti-CMV titer, the purity, the range of distribution of molecular weight, the amount of residue of caprylic acid, and the osmolality of the packed product.

5. The method for preparing intravenous cytomegalovirus human immune globulin, as recited in claim 2, wherein a filling is added in the process of said anion exchange chromatography, wherein said filling is selected from the group consisting of DEAE Sepharose Fast Flow, TOYOPEARL DEAE 650M, and Macro-Prep DEAE Media.
